(19) United States
(12) Patent Application Publication
(10) Pub. No.: US 2019/0225923 A1
(43) Pub. Date: Jul. 25, 2019

(54) APPARATUS FOR EMBRYO BIOPSY

(71) Applicant: Nanyang Technological University, Singapore (SG)

(72) Inventors: Wei Tech ANG, Singapore (SG); Zenan WANG, Singapore (SG)

(21) Appl. No.: 16/337,176

(22) PCT Filed: Sep. 29, 2017
(86) PCT No.: PCT/SG2017/050489

§ 371 Date: Mar. 27, 2019

(30) Foreign Application Priority Data
Sep. 30, 2016 (SG) 10201608177W

(51) Int. Cl. C12M 3/00; H04N 5/232; H04N 5/247; G06T 7/00; H04N 5/225

(52) U.S. Cl. CPC C12M 21/06; H04N 5/23299; H04N 5/247; A61B 10/02; H04N 5/2253; H04N 5/2254; G06T 2207/30044; G06T 7/0012

(57) ABSTRACT

An apparatus for embryo biopsy is provided. The apparatus includes an enclosure and an incubation unit which is disposed in the enclosure and which is configured to incubate an embryo. The apparatus further includes an embryo manipulator setup which is disposed in the enclosure and which is configured to rotate the embryo. The apparatus further includes an embryo image capturing mechanism which is disposed in the enclosure and which is configured to capture an image of the embryo in the incubation unit so as to monitor the morphology of the embryo to determine a development stage of the embryo in the incubation unit. The embryo manipulator setup is further configured to be activated to rotate the embryo to a predetermined orientation based on a determination that the embryo is at a predetermined development stage.

### CROSS-REFERENCE TO RELATED APPLICATIONS

The present application is a national phase entry of PCT/SG2017/050489, filed on Sep. 29, 2017, which claims the benefit of priority of Singapore patent application No. 10201608177W filed on Sep. 30, 2016, the entire contents of which are incorporated herein by reference in their entirety for all purposes.

### TECHNICAL FIELD

Embodiments generally relate to an apparatus for embryo biopsy. In particular, an automated apparatus for embryo biopsy.

### BACKGROUND

Preimplantation genetic diagnosis (PGD) is a procedure which is used for identifying genetic defects within embryos created through in vitro fertilization (IVF) to prevent certain diseases or disorders from being passed on to the child. Intracytoplasmic Sperm Injection (ICIS) is a procedure in which a single sperm is injected directly into an egg. One of the key challenges in procedures such as PGD or (ICIS) is embryo manipulation. Typically, embryo manipulation in a PGD procedure involves a highly skilled operator that uses a micropipette to repeatedly apply vacuum and release the cell until the inner cell mass (ICM) of the embryo is positioned away from any micropipette penetration. This is to ensure that the cell's development competence is preserved for subsequent procedures. Usually, before the operator can work on the embryo, he has to periodically and visually assess the maturity of the embryo that is housed in an incubator every few hours. Conventionally, embryo biopsy for PGD has often been performed on day-three (or three days old) embryos, which is traumatic and lowers the embryo's potential for eventual implantation. On day-three, human embryos usually have about 6 to 10 cells which are larger and have significant cell to cell attachments. But for trophectoderm biopsy, the embryo has to reach a certain stage of maturity, for example at the blastocyst stage between day-five to day-six when the embryo has about 100 cells and clear inner-structure. Usually, there is only a small window between day-five to day-six which is suitable for performing trophectoderm biopsy. Further, the trophectoderm biopsy is also a tedious and laborious process.

### SUMMARY

According to various embodiments, there is provided an apparatus for embryo biopsy. The apparatus may include an enclosure and an incubation unit which may be disposed in the enclosure and which may be configured to incubate an embryo. The apparatus may further include an embryo manipulator setup which may be disposed in the enclosure and which may be configured to rotate the embryo. The apparatus may further include an embryo image capturing mechanism which may be disposed in the enclosure and which may be configured to capture an image of the embryo in the incubation unit so as to monitor the morphology of the embryo to determine a development stage of the embryo in the incubation unit. According to various embodiments, the embryo manipulator setup may be further configured to be activated to rotate the embryo to a predetermined orientation based on a determination that the embryo is at a predetermined development stage.

According to various embodiments, the apparatus may include a biopsy tool. The biopsy tool may be configured to be activated to perform biopsy on the embryo based on a determination that the embryo is at a predetermined orientation.

### BRIEF DESCRIPTION OF THE DRAWINGS

In the drawings, like reference characters generally refer to the same parts throughout the different views. The drawings are not necessarily to scale, emphasis instead generally being placed upon illustrating the principles of the invention. In the following description, various embodiments are described with reference to the following drawings, in which:

FIG. 1 shows a schematic diagram of an apparatus for embryo biopsy according to various embodiments;

FIG. 2 shows a schematic diagram of an apparatus for embryo biopsy according to various embodiments;

FIG. 3A shows a schematic diagram of a partial cut-out perspective view of an apparatus for embryo biopsy according to various embodiments;

FIG. 3B shows a cut-out view of the multi-layer storage rack of the apparatus of FIG. 3A according to various embodiments;

FIG. 4 shows a schematic diagram of a partial cut-out perspective view of an apparatus for embryo biopsy according to various embodiments;

FIG. 5A shows a schematic diagram of an apparatus for embryo biopsy according to various embodiments;

FIG. 5B shows the multi-layer incubator of the apparatus of FIG. 5A according to various embodiments;

FIG. 6 shows a picture of an orientation of an embryo during trophectoderm biopsy according to various embodiments;

FIG. 7 shows a picture illustrating examples of rotating a mouse oocyte according to various embodiments;

FIG. 8 shows an overall sequence of cell rotation according to various embodiments;

FIG. 9 shows schematic diagrams illustrating out-of-plane rotation (FIG. 9 (a) to (c)) according to various embodiments and in-plane rotation (FIG. 9 (d) to (f)) according to various embodiments.

FIG. 10 illustrates a force analysis diagram in the out-of-plane rotation in front view according to various embodiments; and

FIG. 11 illustrates a force analysis diagram in the in-plane rotation in top view according to various embodiments.

### DETAILED DESCRIPTION

Embodiments described below in context of the apparatus are analogously valid for the respective methods, and vice versa. Furthermore, it will be understood that the embodiments described below may be combined, for example, a part of one embodiment may be combined with a part of another embodiment.

It should be understood that the terms “on”, “over”, “top”, “bottom”, “down”, “side”, “back”, “left”, “right”, “front”, “lateral”, “side”, “up”, “down” etc., when used in the following description are used for convenience and to aid understanding of relative positions or directions, and not intended to limit the orientation of any device, or structure or any part of any device or structure. In addition, the singular terms “a”, “an”, and “the” include plural references unless context clearly indicates otherwise. Similarly, the word “or” is intended to include “and” unless the context clearly indicates otherwise.

Various embodiments of an apparatus for embryo biopsy have been provided to address at least some of the issues identified earlier.

According to various embodiments, the apparatus for embryo biopsy may be configured to fully automate the entire process from embryo preparation to embryo biopsy, including but not limited to incubating the embryo, identifying or determining the maturity of the embryo, manipulating the embryo or rotating the embryo into a suitable orientation for biopsy, and conducting the embryo biopsy.

Various embodiments have provided an apparatus for automating embryo biopsy for Preimplantation Genetic Diagnosis (PGD). The apparatus may include an on-stage incubator having a fluidic chamber with controlled temperature and carbon dioxide (CO₂) level for incubating the embryos. The apparatus may further include an imaging system and a processor. The optical imaging system may have a motorized nosepiece configured for tracking embryos housed in the incubator, an autofocusing means to get focused planar images of the embryos, and an image capturing device for capturing the focused planar image. The processor may be configured for further processing the planar image to identify the maturity of the embryo, for example through monitoring the morphology of the embryo including locating and identifying the number/volume/size of blastomeres and/or the presence of a suitably sized inner cell mass (ICM) of the embryo. According to various embodiments, the apparatus may also include an embryo manipulator which may be activatable when the number/volume/size of blastomeres and/or the presence/size of the ICM identified indicates that the embryo is of a suitable maturity and which may be configured to have access to the fluidic chamber of the on-stage incubator. The embryo manipulator may be configured for orienting or rotating the embryo's ICM accordingly (based on the identified position of the ICM) to an operating position where the ICM is not in line of (or in the way of) the penetration of a biopsy tool.

Various embodiments have also provided a method of automatic biopsy of an embryo for Preimplantation Genetic Diagnosis (PGD). The method may include incubating a plurality of embryos in an incubator, continuously having an imaging device capturing images of a planar view of the embryos, and processing the planar images to monitor the morphology of the embryo so as to identify the number/volume/size of blastomeres and/or presence/size of Inner Cell Mass (ICM) in the embryo. The method may further include recording the timing of early cleavage events via the imaging system such that the imaging system may provide details about the culture process, which may help the embryologist to improve the embryo selection. The whole process, development of the embryo culture from day-zero to day-five/day-six may be recorded completely.

Upon identifying the number/volume/size of blastomeres and/or presence/size of ICM, and determining the maturity of the embryo with better embryo selection, the method may include rotating the embryo with the ICM, via allowing access of a manipulating device to the embryo, to an operating position where the ICM is away from the line of (or in the way of) the penetration of the biopsy tool. This may be done through rotating the embryo and using the imaging device to capture the planar view and processing the planar image to verify the location of the ICM .

Further, the method may include holding the embryo stationary and activating the biopsy tool to extract a part of the embryo for further preimplantation diagnosis. The whole procedure of the embryo biopsy may be done with the guide of a vision system which may automate the micromanipulation of the embryo, as well as cutting of the embryo with the biopsy tool and extraction of the part of the embryo.

Subsequently, the extracted part may be sent to designated area for further diagnosis and the embryo may be sent back to the incubator by the control and vision system.

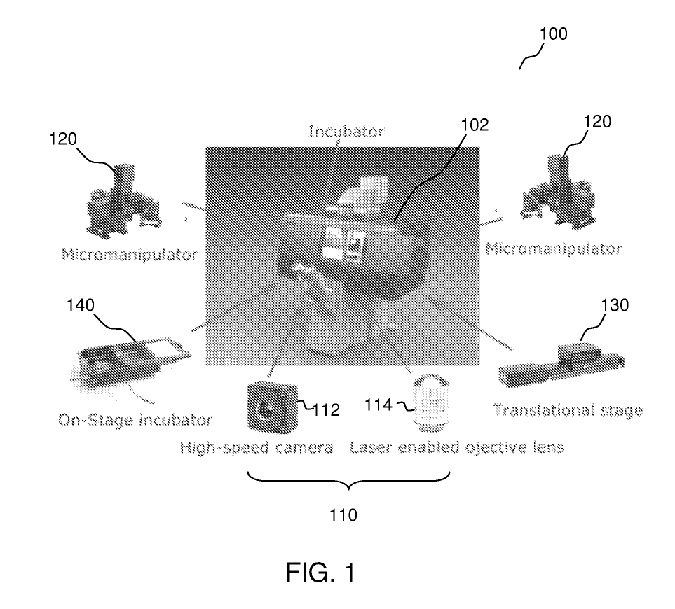

FIG. 1 shows a schematic diagram of an apparatus 100 for embryo biopsy according to various embodiments. The apparatus 100 may be an integrated automated embryo biopsy device. As shown, the apparatus 100 may include the various components as described in the following.

The apparatus 100 may include an image capturing device 110. The image capturing device 110 may include a camera 112 and a laser focusing objective lens 114. The image capturing device 110 may be used to collect images of the embryo and a pipette, wherein the images may be used for embryo monitoring and visual servoing. The images may be processed using an algorithm to identify the maturity of the embryo (for example, monitoring the morphology of the embryo by looking for the ICM or counting the number of blastomeres or measuring the size/volume/dimension of the ICM and/or the blastomeres) and also to monitor the position of the ICM.

The apparatus 100 may further include a manipulator 120. The manipulator 120 may be the component that may be used to rotate and manipulate the embryo. According to various embodiments, the manipulator 120 may be configured to hold a pipette and/or an aspiration pipette. The embryo manipulator 120 may be any device that may be able or may be configurable to rotate the embryo and work in conjunction with the image capturing device 110 which may capture the planar image and detect the position of the ICM. The manipulator 120 may stop when the ICM is detected to be in the correct position (i.e. away from the line of penetration of the biopsy tool).

The apparatus 100 may further include a translational stage 130. The translational stage 130 may be used to open and close an on-stage incubator 140 to maintain the environment (e.g. the temperature and the carbon dioxide level) during biopsy, and/or move the manipulators 120 to access the embryo.

The apparatus 100 may further include the on-stage incubator 140. The on-stage incubator 140 may be used to host the embryos through controlling the carbon dioxide level and temperature when the embryos are under monitoring during incubation. The on-stage incubator may also include heating means or heating mechanism or heating element such that the on-stage incubator may be used as a heat plate when embryo biopsy is taking place.

The apparatus 100 may further include a biopsy tool. The biopsy tool may be used to perform zona cutting and Trophectoderm (TE) cutting. According to various embodiments, the biopsy tool may be laser based or mechanical based biopsy tool. For example, the biopsy tool may be a laser based biopsy device or a physical ultrasonic based cutter. According to various embodiments, the biopsy tool may include a laser source emitting laser through the laser focusing objective lens 114 such that the laser focusing objective lens 114 may focus the laser to breach an outer layer of the embryo while the manipulator 120 may be configured to control a micro-pipette to access the breached area of the embryo so as to extract a part of the embryo.

According to various embodiments, the apparatus 100 may be an all-in-one system that reduces human intervention during the entire process from embryo preparation to embryo biopsy (for example from day-three of incubation to the completion of the biopsy and the extraction of the required part of the embryo on day-five or day-six), not just automation of the cutting and/or extraction for the biopsy process. Accordingly, all operations, including monitoring, recognition, and biopsy, may be completed within the all-in-one system without the need for human/manual intervention. Hence, the apparatus 100 and the method according to various embodiments may be advantageous over conventional Trophectoderm (TE) biopsy.

In order to better understand how the system (or the apparatus 100) and the method according to various embodiments may work in an advantageous manner to perform TE biopsy over conventional TE biopsy, a summary (key steps) of a commonly known conventional TE biopsy protocol is provided in the following as comparison.

Step 1 of the conventional TE biopsy typically involves separate embryo preparation prior to the TE biopsy whereby the fertilized oocyte is stored in a separately provided and independent carbon dioxide (CO₂) incubator.

Step 2 of the conventional TE biopsy typically involves an embryologist manually checking the maturity of the embryo in the CO₂ incubator about once a day during the first three to five days, and at hourly interval on day-five and/or day-six. A clear ICM may typically be identified on day-five or day-six, which signals the readiness of the embryo for TE biopsy. Typically, in the cell development process, usually on Day-zero, there is only one cell. On Day-one, there are two cells. On Day-two, there are four cells. On Day-three, there are eight cells. On Day-five or Day-six, there maybe one or two hundred cells. TE biopsy is usually performed on Day-five or Day-six because this is the time when the embryo cell division may be normal and there may be enough cells. Thus, the embryo during this moment in time may be considered “mature enough”.

Step 3 of the conventional TE biopsy typically involves the embryologist manually taking the embryo out of the CO₂ incubator, and place it on a separate heat plate or another non-CO₂ incubator under the microscope to perform TE biopsy. During the TE biopsy process, the embryo may be required to be manually rotated using micro pipette. This is generally a random process which may be difficult to control. The embryologist may also need to have steady hands and be extremely focused during this process or period. There should also not be any disturbance (e.g. any small vibration that will affect the procedure) during the process.

Accordingly, in comparison to the above conventional TE biopsy process, the apparatus and method according to the various embodiments may be advantageous.

According to various embodiments, the apparatus 100 may include two incubators, which may both be used as CO₂ incubators. For example, the apparatus 100 may be a dual incubator system including an enclosure 102 which may be a main incubator box, and the on-stage incubator 140 placed inside the enclosure 102.

As described above, based on the current commonly used conventional protocol, the embryo must be stored in a separate CO₂ incubator for herniation prior to the biopsy. The embryologist must monitor the maturity of the embryo to determine the suitable time for the biopsy. During biopsy, the embryo must be placed on a heated stage or a non-CO₂ incubator to maintain the correct pH level. Therefore, not only the temperature (37° C.) is required to be maintained, the CO₂ level also has to be maintained at 5% as well. In the conventional manual operation, the embryo must be stored in a fridge-style incubator. The embryologist must also take out the embryo plate frequently to check its maturity. Subsequently, the biopsy is done on a separate heat stage.

In contrast, the apparatus 100 (or the all-in-one system) according to various embodiments may include the enclosure 102 which may be the main incubator and which may enclose the whole system such that both the temperature and the CO₂ level may be controlled and maintained. Inside the enclosure 102 (or the main incubator), a heat element such as on-stage incubator or a heat plate may be additionally installed to maintain the temperature when the enclosure (or the main incubator) is turned off. Therefore, the embryologist may not need to take the embryo plate out of the incubator for testing or switch to a different workstation to perform biopsy.

According to various embodiments, the apparatus 100 (or the main incubator unit) may be operated in various settings, for example, in at least two settings such as a Day-three-to-Day-six setting and a Day-five-to-Day-six setting for embryo biopsy.

According to various embodiments, in the Day-three-to-Day-six setting, on Day-three, upon loading the embryo into the on-stage incubator chamber, the main incubator unit (which is the enclosure 102) and the on-stage incubator 140 may be turned on together with a same environmental setting, i.e. same temperature and same CO₂ percentage level. Both the main incubator unit (or the enclosure 102) and the on-stage incubator 140 may be equipped with an automatically controlled gas inlet and thermostat with electric heating unit. Further, the main incubator unit (or the enclosure 102) and the on-stage incubator 140 may be configured to prevent CO₂ leakage and temperature fluctuation.

Subsequently, on Day-six, once the system (or the apparatus 100) recognizes that the embryo has a clear ICM, which may be an indication that the embryo is mature enough to perform TE biopsy, the main incubator (or the enclosure 102) may be turned off. The temperature and CO₂ level in the main incubator may drop to normal environmental level. Meanwhile, the cover of the on-stage incubator 140 may be opened and the CO₂ supply may be turned off. Further, the temperature may be maintined at 37° C. in the on-stage incubator 140. The apparatus 100 may then be ready for TE biopsy on the embryo. The biopsy may be performed in 37° C. and normal CO₂ level (or non-CO₂) environment. Upon completion of the biopsy, the cover of the on-stage incubator 140 may be closed, and the on-stage incubator 140 may be refilled with CO₂. The main incubator (or the enclosure 102) may also be activated to provide suitable CO₂ level and temperature.

According to various embodiments, in the Day-five-to-Day-six setting, only the on-stage incubator 140 may be turned on when the day-five embryo is load into the apparatus 100. The main incubator unit (or the enclosure 102) may be turned off and may only be used as an enclosure to prevent environmental contamination, i.e. used to establish a clean environment. The rest of the setting may be the same as the Day-three-to-Day-six setting. This is because the on-stage incubator 140 may be sufficient to provide a stable environment for 24 hours. According to various embodiments, the Day-five-to-Day-six biopsy described above may also be performed in the Day-three-to-Day-six setting.

According to various embodiments, the on-stage incubator 140 may include an automatic cover, which may be configured to seal the chamber in the on-stage incubator 140 to maintain the CO₂ and temperature when storing the embryo, as well as facilitate the access to the embryo by automatically opening during biopsy.

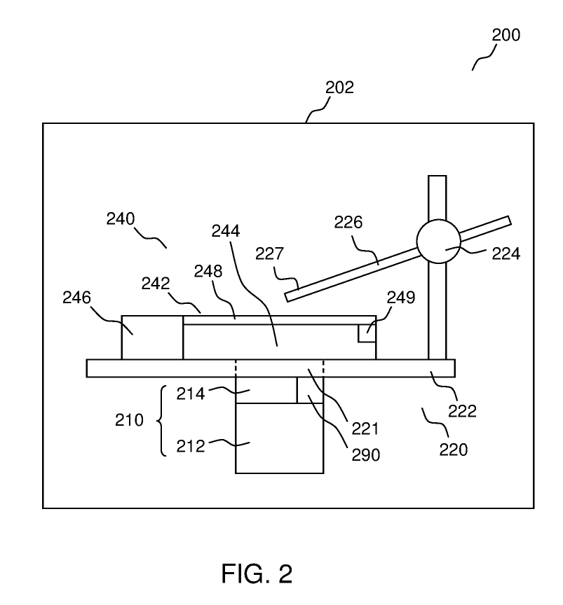

FIG. 2 shows a schematic diagram of an apparatus 200 for embryo biopsy according to various embodiments. As shown, the apparatus 200 may include an enclosure 202. The enclosure 202 may be a box or a container or a case which may be configurable to be fully closed on all sides of the box or the container or the case. According to various embodiments, the enclosure 202 may include a door or cover (not shown) for opening and closing an access or entry into the enclosure 202. The door or cover may be configured to be closed tightly or completely so as to prevent any leakage in order to maintain an environment or condition within the enclosure 202. Accordingly, a space within the enclosure 202 may be sealed off from an external environment such that the space within the enclosure 202 may avoid contamination and may be kept as a clean environment.

As shown in FIG. 2, the apparatus 200 may further include an incubation unit 240. The incubation unit 240 may be disposed in the enclosure 202. The incubation unit 240 may be configured to receive an embryo and to incubate the embryo. According to various embodiments, the incubation unit 240 may be an on-stage incubator 242. The on-stage incubator 242 may include a temperature control mechanism configured to control a temperature inside the on-stage incubator 242, and a carbon dioxide control mechanism configured to control an amount of carbon dioxide inside the on-stage incubator 242. According to various embodiments, the on-stage incubator 242 may include an incubation chamber 244 and a chamber environment control unit 246. The embryo may be directly placed in the incubation chamber 244 or be placed in a dish, such as a petri-dish, which may in turn be placed in the incubation chamber 244. Accordingly, the incubation chamber 244 may contain a fluid medium for embryo growth or the dish may contain the fluid medium. According to various embodiments, the dish may be a multi-well dish and each well may contain one embryo. According to various embodiments, the incubation chamber 244 may include at least one gas inlet (not shown) configured to be in gaseous communication with the chamber environment control unit 246, and a heater element (not shown) configured to be in electrical communication with the chamber environment control unit 246. The chamber environment control unit 246 may include at least one gas supply, for example a mixed air supply and a carbon dioxide supply. The chamber environment control unit 246 may be configured to control a gas mixture flowing into the incubation chamber 244 so as to control a carbon dioxide level within the incubation chamber 244. Further, the chamber environment control unit 246 may further be configured to control a power supply to the heater element so as to control a temperature within the incubation chamber 244. Accordingly, the chamber environment control unit 246 may control the environment of the incubation chamber 244 of the on-stage incubator 242 so as to provide a suitable environment for incubating the embryo.

According to various embodiments, the on-stage incubator 242 may further include a cover 248 and a cover actuator 249 connected to the cover 248. The cover actuator 249 may be configured to actuate the opening and closing of the cover 248. According to various embodiments, the cover 248 may include a sliding cover or a swing cover. Accordingly, the cover actuator 249 may include a linear actuator or a rotary actuator respectively.

As shown in FIG. 2, the apparatus 200 may further include an embryo manipulator setup 220. The manipulator setup 220 may be disposed in the enclosure 202. According to various embodiments, the manipulator setup 220 may be configured to rotate the embryo so as to orientate the embryo in preparation for embryo biopsy. The manipulator setup 220 may also be configured to hold on to the embryo so as to prevent the embryo from movement during embryo biopsy. According to various embodiments, the embryo manipulator setup 220 may include a platform 222 and at least one three-degree-of-freedom micromanipulator 224 attached to the platform 222. The at least one three-degree-of-freedom micromanipulator 224 may be configured to move its end 225 relative to the platform 222. According to various embodiments, a micropipette 226 may be attached to the at least one three-degree-of-freedom micromanipulator 224. The micropipette 226 may be configured to expel or aspirate so as to push or hold on to the embryo. According to various embodiments, the micromanipulator 224 may be configured to move an end of the micropipette 226 into the incubation chamber 244 of the on-stage incubator 242 when the cover 248 of the on-stage incubator 242 is opened.

As shown in FIG. 2, the apparatus 200 may further include an embryo image capturing mechanism 210. The embryo image capturing mechanism 210 may be disposed in the enclosure 202. The embryo image capturing mechanism 210 may be configured to capture an image of the embryo in the incubation unit 240 so as to monitor the morphology of the embryo by identifying the number/volume/size of blastomeres or the presence/size of Inner Cell Mass (ICM) in the embryo in order to determine a development stage of the embryo in the incubation unit 240. According to various embodiments, the image capturing mechanism 210 may be further configured to capture images of the embryo in the incubation unit 240 when the embryo manipulator setup 220 is rotating the embryo so as to monitor the position of the inner cell mass to determine an orientation of the embryo.

According to various embodiments, the image capturing mechanism 210 may be attached to the platform 222 of the manipulator setup 220. As shown, the image capturing mechanism 210 may be attached to the platform 222 from underneath and the on-stage incubator 242 may be on the platform 222. According to various embodiments, the image capturing mechanism 210 may include an imaging device 212, such as a camera, and an objective lens 214. As shown, the objective lens 214 may be arranged between the on-stage incubator 242 and the imaging device 212. Accordingly, the platform 222 may include an opening or a transparent portion 221 for the image capturing mechanism 210, and the on-stage incubator 242 may include a transparent portion at the base such that the image capturing mechanism 210 may obtain images of the embryo in the on-stage incubator 242 during incubation of the embryo, during rotation of the embryo in preparation for embryo biopsy, and during embryo biopsy.

According to various embodiments, the manipulator setup 220 may be further configured to be activated to rotate the embryo to a predetermined orientation based on a determination that the embryo is at a predetermined development stage. Accordingly, during incubation of the embryo in the on-stage incubator 242, the image capturing mechanism 210 may capture image of the embryo for monitoring the morphology of the embryo by identifying the number/volume/size of blastomeres or the presence/size of the ICM so as to determine the development stage of the embryo. Once the embryo is determined to be of the predetermined desired development stage suitable for embryo biopsy, the manipulator setup 220 may be activated to actuate the at least one three-degree-of-freedom micromanipulator 224 so as to move the micropipette 226 for accessing the embryo in the incubation chamber 244 of the on-stage incubator 242. The cover actuator 249 of the on-stage incubator 242 may also be configured to be activated to actuate the opening of the cover 248 based on the determination that the embryo in the on-stage incubator 242 is at the predetermined development stage such that the micropipette 226 may access into the on-stage incubator 242. When the cover 248 of the on-stage incubator 242 is opened, the on-stage incubator 242 may be configured to maintain a temperature of the embryo within the on-stage incubator 242. For example, with the heater element at the base of the on-stage incubator 242.

According to various embodiments, the enclosure 202 may be an incubator box. Accordingly, when the cover 248 of the on-stage incubator 242 is opened, the enclosure 202 in the form of the incubator box may still control the gas composition, such as the carbon dioxide level, and the temperature within the enclosure 202.

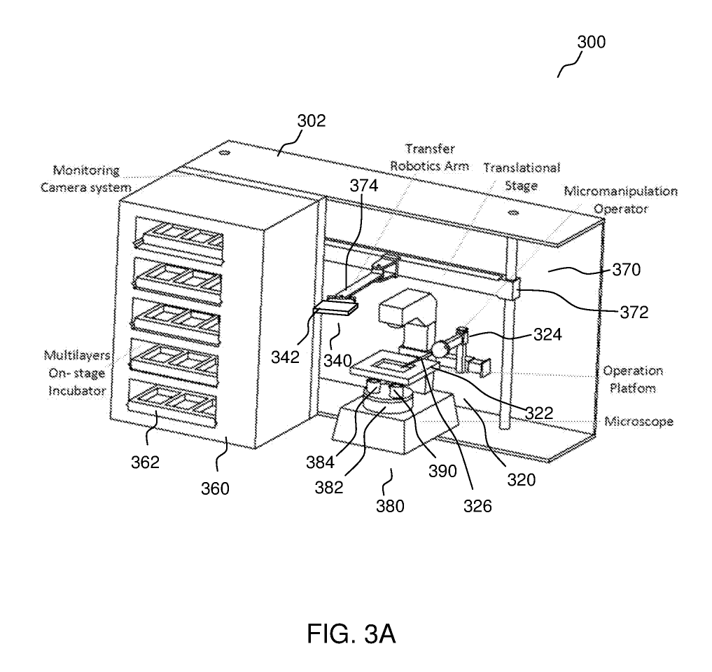

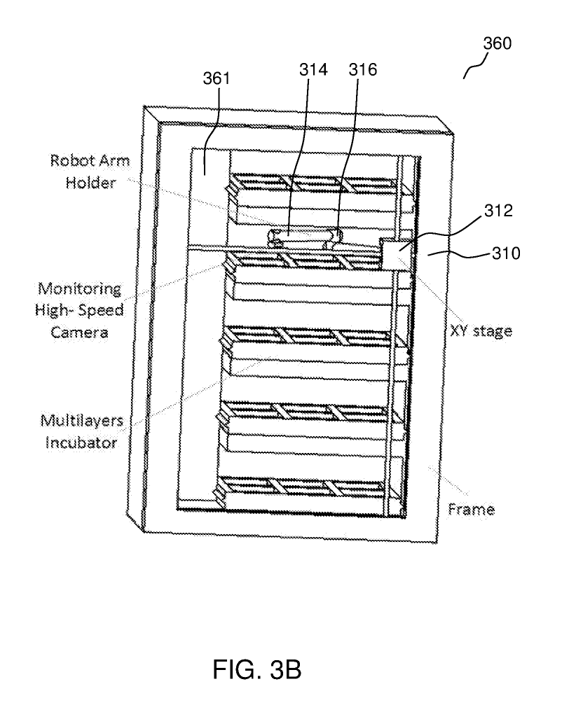

FIG. 3A shows a schematic diagram of a partial cut-out perspective view of an apparatus 300 for embryo biopsy according to various embodiments. The apparatus 300 may, similar to the apparatus 200 of FIG. 2, include an enclosure 302. The enclosure 302 may be similar to the enclosure 202 of the apparatus 200 of FIG. 2. The apparatus 300 may, similar to the apparatus 200 of FIG. 2, further include an embryo manipulator setup 320 disposed in the enclosure 302. The manipulator setup 320 may be similar to the manipulator setup 220 of the apparatus 200 of FIG. 2 and may be configured to rotate the embryo so as to orientate the embryo in preparation for embryo biopsy. Accordingly, the manipulator setup 320 may include a platform 322 and at least one three-degree-of-freedom micromanipulator 324 attached to the platform 322. Further, a micropipette 326 may be attached to the at least one three-degree-of-freedom micromanipulator 324.

According to various embodiments, the apparatus 300 may further include a plurality of incubation unit 340. The plurality of incubation unit 340 may be disposed in the enclosure 302. Each of the incubation unit 340 may be an on-stage incubator 342 and may be configured to incubate an embryo. Each of the on-stage incubator 342 may be similar to the on-stage incubator 242 of the apparatus 200 of FIG. 2. Accordingly, the on-stage incubator 342 may include an incubation chamber and a chamber environment control unit. The on-stage incubator 342 may further include a cover and a cover actuator connected to the cover. The cover actuator may be configured to actuate the opening and closing of the cover. Each on-stage incubator 242 may individually control a respective temperature and carbon dioxide concentration level.

As shown in FIG. 3A, the apparatus 300 differs from the apparatus 200 of FIG. 2 in that the apparatus 300 may include a multi-layer storage rack 360. The multi-layer storage rack 360 may be disposed in the enclosure 302. Further, the multi-layer storage rack 360 may be configured to store the plurality of on-stage incubators 342. The multi-layer storage rack 360 may be configured such that each layer may receive a tray 362, and each tray 362 may receive a pre-determined number of on-stage incubators 342. The multi-layer storage rack 360 may be configured to provide a dark environment for the plurality of on-stage incubators 342 to facilitate incubation of the embryos.

As shown in FIG. 3A, the apparatus 300 may further include a transfer mechanism 370 disposed in the enclosure 302. The transfer mechanism 370 may be configured to retrieve one of the plurality of on-stage incubators 342 from the multi-layer storage rack 360 and to place the one of the plurality of on-stage incubators 342 on the platform 322. The transfer mechanism 370 may include a motorized two-axis translation transfer stage 372 and a three-degree-of-freedom transfer robotic arm 374 attached to the motorized two-axis translation transfer stage 372. The motorized two-axis translation transfer stage 372 may be configured to move the three-degree-of-freedom transfer robotic arm 374 between the multi-layer storage rack 360 and the manipulator setup 320. Further, the three-degree-of-freedom robotic arm 374 may be configured to reach into the multi-layer storage rack 360 to pick up the one of the plurality of on-stage incubators 342 from the multi-layer storage rack 360 and to move and position the one of the plurality of on-stage incubators 342 onto the platform 322 of the manipulator setup 320. Accordingly, the transfer robotic arm 374 may grab and hold a chosen on-stage incubator 342 from the multi-layer storage rack 360 and move it to the platform 322 of the manipulator setup 320. According to various embodiments, the motorized two-axis translation transfer stage 372 may include a linear guide rail and a guide screw. According to various embodiments, the three-degree-of-freedom transfer robotic arm 374 may be configured to grab any desired on-stage incubator 342 from anywhere in the multi-layer storage rack 360. Further, the rotational joint at the end effector of the three-degree-of-freedom transfer robotic arm 374 may be configured to set the orientation of an on-stage incubator 342 when placing the on-stage incubator 342 on the platform 322 of the manipulator setup 320. Accordingly, the transfer robotic arm 374 may be configured to fold over a range of angles and to move along the linear guide rail in a horizontal direction and along the guide screw in a vertical direction.

According to various embodiments, the platform 322 may include a recess or a cubic space configured for receiving the on-stage incubator 342. Accordingly, the recess may be shaped and sized to correspond with the on-stage incubator 342 such that the on-stage incubator 342 may be placed accurately on the platform 322.

FIG. 3B shows a cut-out view of the multi-layer storage rack 360 of the apparatus 300 according to various embodiments. As shown, the apparatus 300 may further include an embryo image capturing mechanism 310. The embryo image capturing mechanism 310 may be disposed in the enclosure 302 and adjacent to the multi-layer storage rack 360. The embryo image capturing mechanism 310 may be configured to capture an image of the embryo in the incubation unit 340 so as to monitor the morphology of the embryo by identifying the number/volume/size of blastomeres or the presence/size of Inner Cell Mass (ICM) in the embryo to determine a development stage of the embryo in the incubation unit 340. According to various embodiments, the embryo image capturing mechanism 310 may be configured to capture image of the embryo in each of the plurality of on-stage incubators 342. As shown, the embryo image capturing mechanism 310 may include a motorized two-axis translation imaging stage 312, for example a X-Y axis movement stage, disposed in the enclosure 302 and adjacent to the multi-layer storage rack 360. The embryo image capturing mechanism 310 may further include a two-degree-of-freedom imaging robotic arm 314 attached to the motorized two-axis translation imaging stage 312. The embryo image capturing mechanism 310 may further include an imaging device 316, such as a camera or a high speed camera, attached to an end of the two-degree-of-freedom imaging robotic arm 314. According to various embodiments, the motorized two-axis translation imaging stage 312 may be configured to move the two-degree-of-freedom imaging robotic arm 314 between different layers of the multi-layer storage rack 360 and across a width of each layer of the multi-layer storage rack 360. Further, the two-degree-of-freedom imaging robotic arm 314 may be configured to move the imaging device 316 within each layer of the multi-layer storage rack so as to reach out to each on-stage incubator 342 on each layer in order to obtain images of the embryo in each on-stage incubator 342. Accordingly, the imaging device 316 held by the two-degree-of-freedom imaging robotic arm 314 may be moved above or on top of each on-stage incubator 342 in each layer of the multi-layer storage rack 360 so as to obtain images of the embryos inside each of the on-stage incubator 342. Further, the imaging robotic arm 314 may be moved from layer by layer via the motorized two-axis translation imaging stage 312. According to various embodiments, when the two-degree-of-freedom imaging robotic arm 314 is fully folded, the two-degree-of-freedom imaging robotic arm 314 may be moved up and down through the alleyway 361 at a corner in the multi-layer storage rack 360. According to various embodiments, the embryo image capturing mechanism 310 may be able to monitor the embryos' development or morphology when the embryos are incubating.

According to various embodiments, for monitoring the embryo's morphology or development, the embryo image capturing mechanism 310 may include a light source with a special wave length, a digital inverted microscope and some other optic modules for improving the contrast. During the culture period (for example from Day-one to Day-five/six), except the time for capturing image, the embryo may be maintained in a dark environment within the multi-layer storage rack 360. During image capturing, the time for light exposure may be about 0.04s which may be very short and may be just a twinkling or an instant. The digital inverted microscope may be customized with proper optic lens and a digital micro camera. This may help to reduce the size of imaging device 316. Accordingly to various embodiments, two kinds of microscopy methods, namely dark field and bright field, may be possible. Dark field illumination may allow more accurate observations of the blastomere membrane and may provide more accurate information about cleavage. However, it may sacrifice some details about intracellular morphology beyond Day-two. On the other hand, bright field illumination is a straightforward microscopy method which may allow observation of dark sample on a bright background. Bright field may be commonly used for stained or naturally pigmented or highly contrasted specimens.

According to various embodiments, based on the determination through the embryo image capturing mechanism 310 that the embryo in the on-stage incubator 342 is at the predetermined development stage, the transfer mechanism 370 may be further configured to retrieve the on-stage incubator 342 from the multi-layer storage rack 360 and to place the on-stage incubator 342 on the platform 322. Further, the cover actuator of the on-stage incubator 342 may be configured to be activated to actuate the opening of the cover, and the embryo manipulator setup 320 may be further configured to be activated to rotate the embryo to a predetermined orientation.

Referring back to FIG. 3A, the apparatus 300 may further include an auxiliary image capturing mechanism 380 disposed in the enclosure 302. The auxiliary image capturing mechanism 380 may be attached to the platform 322 of the manipulator setup 320. The auxiliary image capturing mechanism 380 may include an auxiliary imaging device 382 and an objective lens 384. The objective lens 384 may be arranged between the platform 322 of the manipulator setup 320 and the auxiliary imaging device 382. According to various embodiments, the auxiliary image capturing mechanism 380 may be further configured to capture images of the embryo when the embryo manipulator setup 320 is rotating the embryo in the on-stage incubator placed on the platform so as to monitor the position of the inner cell mass to determine an orientation of the embryo. Accordingly, the platform 322 may include an opening or a transparent portion for the auxiliary image capturing mechanism 380, and the on-stage incubator 342 may include a transparent portion at the base such that the image capturing mechanism 380 may obtain images of the embryo in the on-stage incubator 342 during rotation of the embryo in preparation for embryo biopsy, and during embryo biopsy.

According to various embodiments, after localization or the determination through the embryo image capturing mechanism 310 that the embryo in the on-stage incubator 342 is at the predetermined development stage, the manipulator setup 320 may be operated or worked under the guidance of the vision feedback provided by the auxiliary image capturing mechanism 380. Based on an advanced image processing algorithm, the auxiliary image capturing mechanism 380 and the manipulator setup 320 may be auto-controlled with function like auto-focusing, auto-recognition and positioning the embryo. According to various embodiments after the embryo is rotated to an appropriate and desired orientation, a biopsy tool in the form of a prepared laser may automatically cut the zona pellucida (ZP) layer of the embryo and extract some cells for further diagnosis. After that, the transfer mechanism 370 may be configured to bring the on-stage incubator 342 back to the original location in the multi-layer storage rack 360. According to various embodiments, during the transfer process of the on-stage incubator 342, the on-stage incubator 342 may control the temperature and the carbon dioxide concentration level of on-stage incubator 342.

According to various embodiments, the enclosure 302 may be an incubator box. Accordingly, when the cover of the on-stage incubator 342 is opened, the enclosure 302 in the form of the incubator box may still control the gas composition, such as the carbon dioxide level, and the temperature within the enclosure 302.

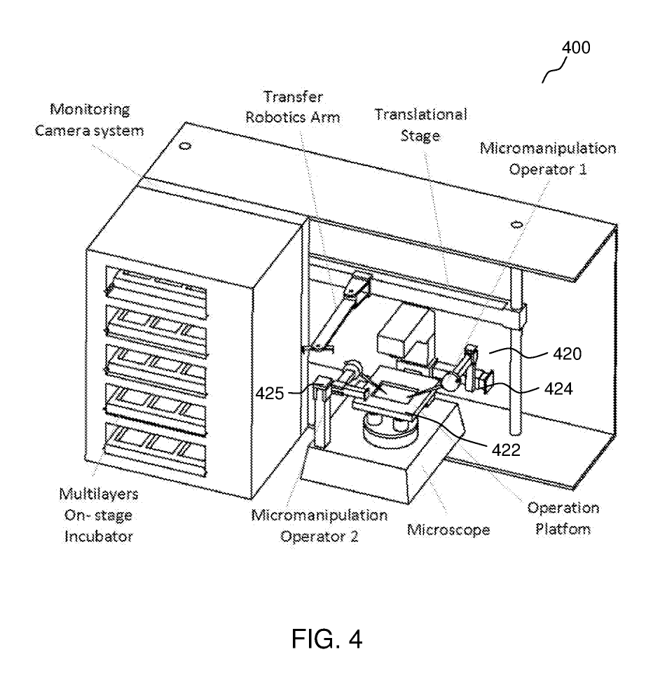

FIG. 4 shows a schematic diagram of a partial cut-out perspective view of an apparatus 400 for embryo biopsy according to various embodiments. The apparatus 400 differs from the apparatus 300 of FIG. 3A in that the embryo manipulator setup 420 include two three-degree-of-freedom micromanipulator 424, 425 attached to a platform 422. The other components of the apparatus 400 are similar to the corresponding components in the apparatus 300 of FIG. 3A. According to various embodiments, the apparatus 400 may include a holding micropipette attached to one of the two three-degree-of-freedom micromanipulator 424 and an injection pipette attached to the other one of the two three-degree-of-freedom micromanipulator 425.

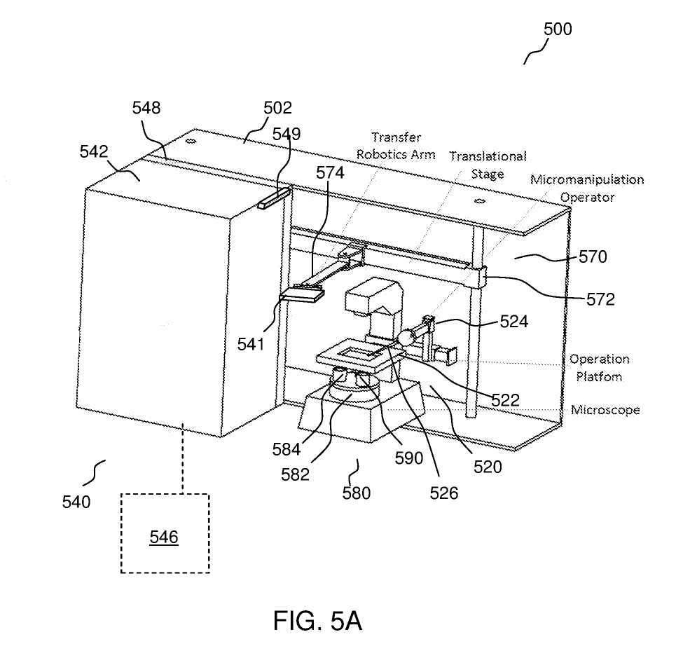

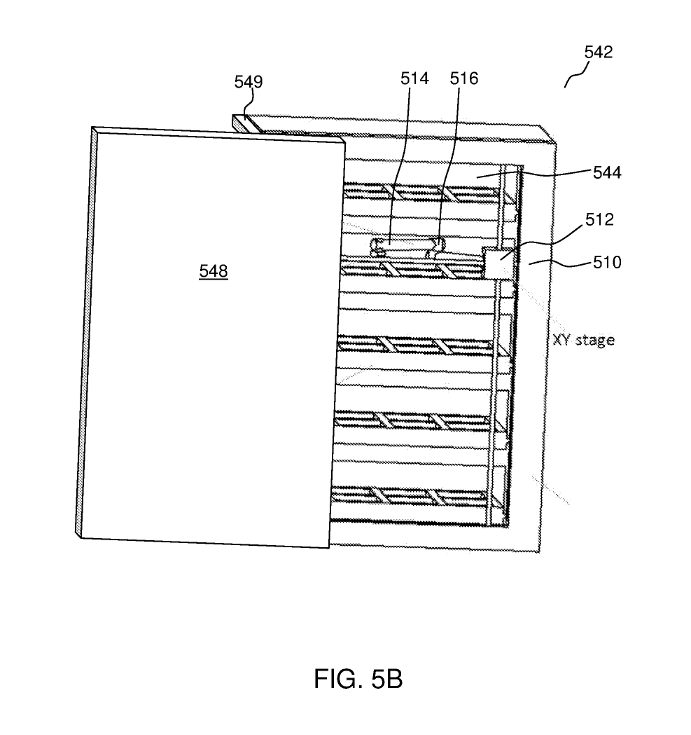

FIG. 5A shows a schematic diagram of an apparatus 500 for embryo biopsy according to various embodiments. The apparatus 500 may, similar to the apparatus 200 of FIG. 2 or the apparatus 300 of FIG. 3A, include an enclosure 502. The enclosure 502 may be similar to the enclosure 202 of the apparatus 200 of FIG. 2 or the enclosure 302 of the apparatus 300 of FIG. 3A. The apparatus 500 may, similar to the apparatus 200 of FIG. 2 or the apparatus 300 of FIG. 3A, further include an embryo manipulator setup 520 disposed in the enclosure 502. The manipulator setup 520 may be similar to the manipulator setup 220 of the apparatus 200 of FIG. 2 or the manipulator setup 320 of the apparatus 300 of FIG. 3A, and may be configured to rotate the embryo so as to orientate the embryo in preparation for embryo biopsy. The manipulator setup 520 may also be configured to hold on to the embryo during embryo biopsy. Accordingly, the manipulator setup 520 may include a platform 522 and at least one three-degree-of-freedom micromanipulator 524 attached to the platform 522. Further, a micropipette 526 may be attached to the at least one three-degree-of-freedom micromanipulator 524.

As shown in FIG. 5A, the apparatus 500 may further include an incubation unit 540. The incubation unit 540 may be disposed in the enclosure 502. The incubation unit 540 may be a multi-layer incubator 542 configured to store a plurality of embryo containers 541. FIG. 5B shows the multi-layer incubator 542 of the apparatus 500 of FIG. 5A. According to various embodiments, the embryo containers 541 may include dishes such as petri-dishes or multi-well dishes. An embryo may be hosted in an embryo container 541. The embryo containers may be configured to be of any suitable shapes and/or sizes.

According to various embodiments, the multi-layer incubator 542 may include a temperature control mechanism configured to control a temperature inside the multi-layer incubator 542, and a carbon dioxide control mechanism configured to control an amount of carbon dioxide inside the multi-layer incubator 542. According to various embodiments, the multi-layer incubator 542 may include a main incubation chamber 544 with racks or trays for holding the plurality of embryo container 541. The multi-layer incubator 542 may also include a main chamber environment control unit 546. According to various embodiments, the main incubation chamber 544 may include at least one gas inlet (not shown) configured to be in gaseous communication with the main chamber environment control unit 546, and a heater element (not shown) configured to be in electrical communication with the main chamber environment control unit 546. The main chamber environment control unit 546 may include or may be connected to at least one gas supply tank, for example a mixed air supply tank and a carbon dioxide supply tank. The main chamber environment control unit 546 may be configured to control a gas mixture flowing into the main incubation chamber 544 so as to control a carbon dioxide level within the main incubation chamber 544. Further, the main chamber environment control unit 546 may further be configured to control a power supply to the heater element so as to control a temperature within the main incubation chamber 544. Accordingly, the main chamber environment control unit 546 may control the environment of the main incubation chamber 544 of the multi-layer incubator 542 so as to provide a suitable environment for incubating the embryo.

According to various embodiments, the multi-layer incubator 542 may further include a door 548 and a door actuator 549 connected to the door 548. The door actuator 549 may be configured to actuate the opening and closing of the door 548. According to various embodiments, the door 548 may include a sliding door or a swing door. Accordingly, the door actuator 549 may include a linear actuator or a rotary actuator respectively.

The apparatus 500 may, similar to the apparatus 300 of FIG. 3A, include a transfer mechanism 570 disposed in the enclosure 502. The transfer mechanism 570 may be configured to retrieve one of the plurality of embryo containers 541 from the multi-layer incubator 542 and to place the one of the plurality of embryo containers 541 on the platform 522 of the manipulator setup 520. Similar to the transfer mechanism 370 of FIG. 3A, the transfer mechanism 570 may also include a motorized two-axis translation transfer stage 572 and a three-degree-of-freedom transfer robotic arm 574 attached to the motorized two-axis translation transfer stage 572. The motorized two-axis translation transfer stage 572 may be configured to move the three-degree-of-freedom transfer robotic arm 574 between the multi-layer incubator 542 and the manipulator setup 520. Further, the three-degree-of-freedom robotic arm 574 may be configured to reach into the multi-layer incubator 542 to pick up the one of the plurality of embryo containers 541 from the multi-layer incubator 542 and to move and position the one of the plurality of embryo containers 541 onto the platform 522 of the manipulator setup 520. Accordingly, the transfer robotic arm 574 may grab and hold a chosen embryo container 541 from the multi-layer incubator 542 and move it to the platform 522 of the manipulator setup 520. According to various embodiments, the three-degree-of-freedom transfer robotic arm 374 may be configured to grab any desired embryo container 541 from anywhere in the multi-layer incubator 542. Further, the end effector of the three-degree-of-freedom transfer robotic arm 574 may be configured to set the orientation of embryo container 541 when placing the embryo container on the platform 522 of the manipulator setup 520.

The apparatus 500 may, similar to the apparatus 300 of FIG. 3A, further include an embryo image capturing mechanism 510 disposed in the enclosure 502. The embryo image capturing mechanism 510 may be configured to capture images of the embryos in the incubation unit 540 in the form of the multi-layer incubator 542 so as to monitor the morphology of the embryos by identifying the number/volume/size of blastomeres or the presence/size of Inner Cell Mass (ICM) in the respective embryo to determine a development stage of the respective embryo in the incubation unit 540. As shown, the embryo image capturing mechanism 510 may be configured to capture image of the embryo in each of the plurality of embryo containers stored in the multi-layer incubator 542. As shown, the embryo image capturing mechanism 510 may include a motorized two-axis translation imaging stage 512 disposed in the multi-layer incubator 542. The embryo image capturing mechanism 510 may further include a two-degree-of-freedom imaging robotic arm 514 attached to the motorized two-axis translation imaging stage 512. The embryo image capturing mechanism 510 may further include an imaging device 516, such as a camera or a high speed camera, attached to an end of the two-degree-of-freedom imaging robotic arm 514. According to various embodiments, the motorized two-axis translation imaging stage 512 may be configured to move the two-degree-of-freedom imaging robotic arm 514 between different layers of the multi-layer incubator 542 and across a width of each layer of the multi-layer incubator 542. Further, the two-degree-of-freedom imaging robotic arm 514 may be configured to move the imaging device 516 within each layer of the multi-layer incubator 542 so as to reach into each layer in order to obtain images of the embryo in each embryo container in the multi-layer incubator 542. Accordingly, the imaging device 516 held by the two-degree-of-freedom imaging robotic arm 514 may be moved above or on top of each embryo container in each layer of the multi-layer incubator 542 so as to obtain images of the embryos inside each of the embryo container. Further, the imaging robotic arm 514 may be moved from layer by layer via the motorized two-axis translation imaging stage 512. According to various embodiments, the embryo image capturing mechanism 510 may be able to monitor the embryos' development or morphology when the embryos are incubating in the multi-layer incubator 542.

According to various embodiments, based on the determination through the embryo image capturing mechanism 510 that the embryo in the embryo container is at the predetermined development stage, the door actuator of the multi-layer incubator 542 may be configured to be activated to actuate the opening of the door. Further, the transfer mechanism 570 may be further configured to be activated to retrieve the embryo container from the multi-layer incubator 542 and to place the embryo container on the platform 522 of the manipulator setup 520. Furthermore, the embryo manipulator setup 520 may be further configured to be activated to rotate the embryo to a predetermined orientation.

According to various embodiments, the apparatus 500 may further include an auxiliary image capturing mechanism 580 disposed in the enclosure 502. The auxiliary image capturing mechanism 580 may be similar to the auxiliary image capturing mechanism 380 of the apparatus 300 of FIG. 3A. Accordingly, the auxiliary image capturing mechanism 580 may be attached to the platform 522 of the manipulator setup 520. The auxiliary image capturing mechanism 580 may include an auxiliary imaging device 582 and an objective lens 584. The objective lens 584 may be arranged between the platform 522 of the manipulator setup 520 and the auxiliary imaging device 582. According to various embodiments, the auxiliary image capturing mechanism 580 may be further configured to capture images of the embryo when the embryo manipulator setup 520 is rotating the embryo in the embryo container placed on the platform 522 so as to monitor the position of the inner cell mass to determine an orientation of the embryo. Accordingly, the platform 522 may include an opening or a transparent portion and the embryo container may include a transparent portion at the base such that the image capturing mechanism 580 may obtain images of the embryo in the embryo container during rotation of the embryo in preparation for embryo biopsy, and during embryo biopsy.

According to various embodiments, the enclosure 502 may be an incubator box. Accordingly, when the door 548 of the multi-layer incubator 542 is opened, the enclosure 502 in the form of the incubator box may still control the gas composition, such as the carbon dioxide level, and the temperature within the enclosure 502.

According to various embodiments, each of the apparatus 100, 200, 300, 400, 500 as described herein may include a biopsy tool (e.g. 290, 390, 590) which may be attached to the respective embryo manipulator setup. The biopsy tool may be configured to extract a part of the embryo. According to various embodiments the biopsy tool may be a laser-based tool or a mechanical based tool.

According to various embodiments, the biopsy tool (e.g. 290, 390, 590) may be activated to perform biopsy on the embryo based on a determination that the embryo is at a predetermined orientation. The biopsy performed may include cutting/breaching an outer layer (e.g. zona pellucida layer) of the embryo and extracting a part of the embryo from within. Referring to FIG. 2, FIG. 3A and FIG. 5A, the image capturing mechanism 210 or the auxiliary image capturing mechanism 380, 580 may be configured to provide visual feedback for monitoring the orientation of the embryo during rotation of the embryo by the embryo manipulator setup 220, 320, 520. Accordingly, the embryo manipulator setup 220, 320, 520 may continuously rotate the embryo until a detected relative position of the inner cell mass of the embryo by the image capturing mechanism 210 or the auxiliary image capturing mechanism 380, 580 is at a predetermined relative position which indicates that the embryo is at the predetermined orientation. Once the image capturing mechanism 210 or the auxiliary image capturing mechanism 380, 580 determines that the embryo is at the predetermined orientation, the respective biopsy tool and/or embryo manipulator setup 220, 320, 520 may be activated to perform embryo biopsy which may include automated cutting/breaching of the embryo and automated extraction of a part of the embryo.

According to various embodiments, each of the apparatus 100, 200, 300, 400, 500 as described herein may also include a processer configured to receive the captured image from the respective embryo image capturing mechanism 110, 210, 380, 580, and to process the captured image to identify the presence/size of ICM or the number/volume/size of blastomeres so as to determine a development stage of the embryo. The processor may also be configured to control the respective embryo manipulator setup 120, 220, 320, 420, 520 to rotate the embryo into a predetermined orientation upon the determination that the embryo is at a predetermined development stage. The processor may also be configured to control various other aspect of the apparatus 100, 200, 300, 400, 500, for example such as opening and closing of the cover of the on-stage incubator 342, opening and closing of the door of the multi-layer incubator 542, controlling the temperature and carbon dioxide concentration level in the respective incubators, transferring of the on-stage incubator or the embryo container, moving of the imaging device, operating the biopsy tool to perform embryo biopsy. The processor may be understood as any kind of a logic implementing entity, which may be special purpose circuitry or a processor executing software stored in a memory, firmware, or any combination thereof. Thus, the processor may be a hard-wired logic circuit or a programmable logic circuit such as a programmable processor, e.g. a microprocessor (e.g. a Complex Instruction Set Computer (CISC) processor or a Reduced Instruction Set Computer (RISC) processor). The processor may also be a processor executing software, e.g. any kind of computer program, e.g. a computer program using a virtual machine code such as e.g. Java. According to various embodiments, the processor may be integrated in the respective apparatus of the various embodiments or may be a separate device connected to the respective apparatus of the various embodiments.

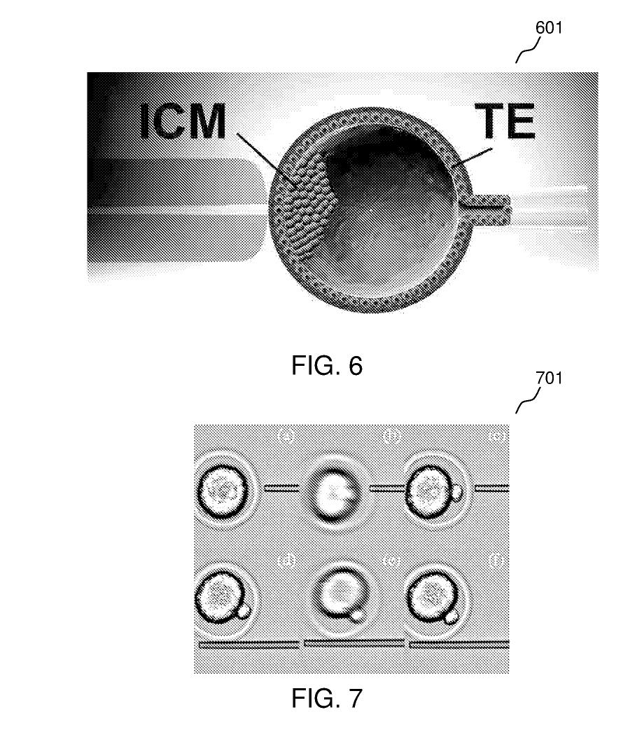

According to various embodiments, the orientation of an embryo may be critical during embryo biopsy. For example, the inner cell mass (ICM), which will grow into fetus eventually, must be kept away from the incision of the zona pellucida or biopsy tool tip to avoid damage of the ICM, as shown in FIG. 6. FIG. 6 shows a picture 601 of an orientation of an embryo during trophectoderm biopsy.

Various embodiments of the apparatus as described herein are configured in a manner whereby the apparatus may be able to automate the biopsy process. For example, the apparatus according to the various embodiments may include a holding micropipette and an injection pipette installed on a pair of three-degree-of-freedom micromanipulators, a motorized X-Y-Z translational stage, and a microinjector. In addition, a high-speed camera may be installed to perform image capturing. It is noted that other rotational means or control method/system/apparatus may be deployed to rotate the embryo to the operating location without departing from the concept or the scope of the embodiments as described herein.

According to various embodiments, the apparatus/system may be able to detect the ICM and rotate the cell to a desired position. FIG. 7 shows a picture 701 illustrating examples of rotating a mouse oocyte, which has a very similar size to the human embryo, using the apparatus/system according to the various embodiments. As shown, the apparatus/system according to the various embodiments may be able to rotate the cell to a desired position in both horizontal and vertical plane using the method described in the following, including out-of-plane rotation and in-plane rotation. In FIG. 7, (a) to (c) illustrate out-of-plane rotation of the mouse oocyte, and (d) to (f) illustrate in-plane rotation of a mouse oocyte.

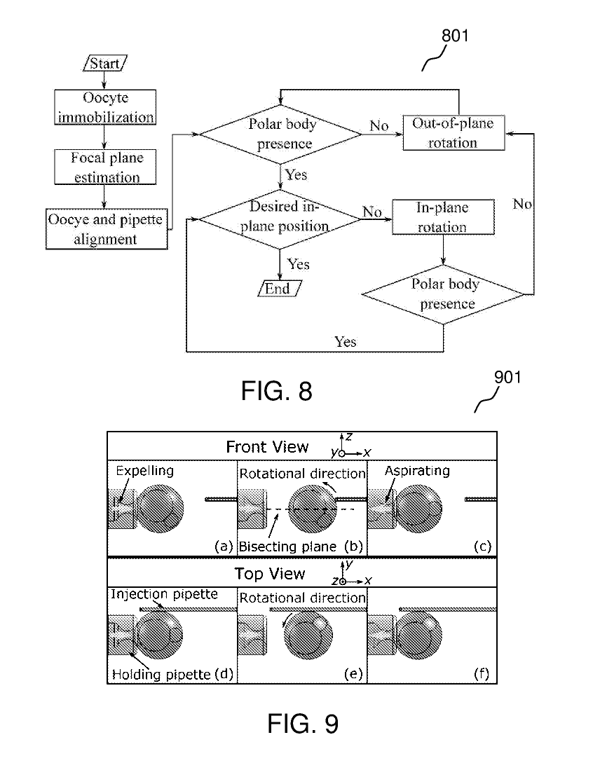

FIG. 8 shows an overall sequence 801 of cell rotation according to various embodiments.

FIG. 9 shows schematic diagrams 901 illustrating out-of-plane rotation (FIG. 9 (a) to (c)) according to various embodiments and in-plane rotation (FIG. 9 (d) to (f)) according to various embodiments.

Out-of-plane Rotation: Similar to the method used in manual manipulation, the out-of-plane rotation may be based on a trial-and-error approach. The pressure at the tip of the holding micropipette may be about ±8 in H2O (or 1992.71 Pa), depending on the applications (aspirating or expelling). Various experimental results suggest that 8 to 10 in H2O (or 1992.71 Pa to 2488.2 Pa) pressure is suitable to the application. FIG. 9(a)-(c) illustrate the front view of the oocyte. Initially, the polar body may not be detected in the image/bisecting plane. The microinjector may generate positive pressure to expel the oocyte at the pipette tip. The expelling force may push the oocyte towards the injection micropipette, which may be about 10 μm away from the oocyte in the x-axis direction and 20 μm above the bisecting plane in the z-axis direction. The duration of applying positive pressure at the tip may be about 10 μs, after which the oocyte may be aspirated back to the holding pipette tip rapidly.

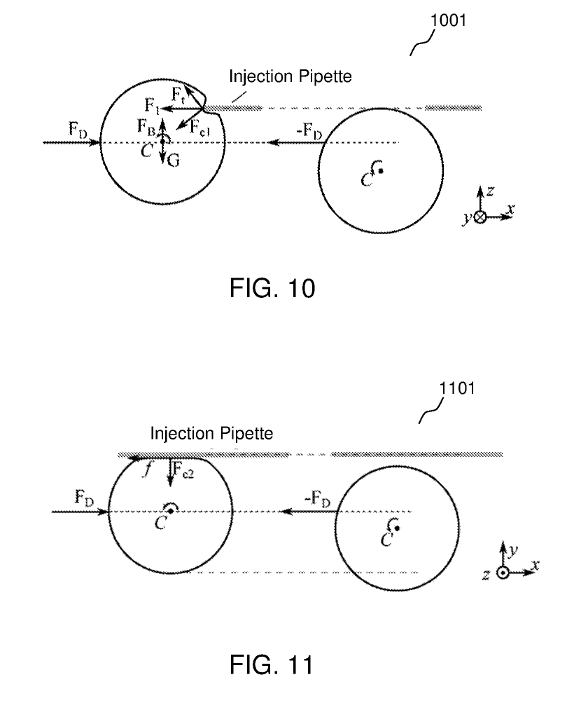

FIG. 10 illustrates a force analysis diagram 1001 in the out-of-plane rotation in front view according to various embodiments. As shown in FIG. 10, as the oocyte moves along its x-axis, it may be blocked at its 2 o'clock position (front view). There may be two types of forces that facilitate the rotation of the oocyte, the tangential force, Fₜ, at the contact point, and the dragging force −F_{D} at its 10 o'clock position. When the oocyte is in contact with the injection pipette, Ft generates a torque that induces the counter-clockwise rotation of the oocyte. Meanwhile, F_{c1} pushes the oocyte downward, which deviates its centre from the dragging force, −F_{D}. The torque generated by −F_{D} leads to further counter-clockwise rotation of the oocyte. The oocyte may be expelled and aspirated repeatedly. Once the polar body is visible in the image plane, the out-of-plane rotation may be stopped.

In-plane Rotation: Upon completion of the out-of-plane rotation, the apparatus/system may perform the in-plane rotation based on the position of the polar body in the image plane. FIG. 9(d)-(f) demonstrate the top view of the oocyte and micropipettes. For instance, the polar body in FIG. 9(d) may be detected to be at 3 o'clock position. In order to perform counter-clockwise rotation, the injection micropipette may be positioned in the focal plane of the oocyte to ensure a firm contact with the zona pellucida, thus to induce enough frictional force. Before pushing the oocyte forward along its x axis by the positive pressure from the holding pipette, the oocyte may be pressed down by the injection micropipette in the y-axis direction. FIG. 11 illustrates a force analysis diagram 1101 in the in-plane rotation in top view according to various embodiments. When the microinjector generates expelling force, the oocyte may be pushed out from the holding pipette. The contact between the oocyte and the injection micropipette may induce a normal force, F_{c2} and frictional force, f, as shown in FIG. 11. The frictional force may rotate the oocyte in counter-clockwise direction before it diminishes as the cell is pushed away by F_{c2}. When the oocyte's centre is off the axis of −F_{D}, the dragging force may further induce the rotation of the oocyte in counter-clockwise direction. The position of the polar body may be monitored at all time to determine if the polar body is rotated to a desired position. Due to gravity, the oocyte may experience the out-of-plane rotation, but as the rotation period is considerably short, and the oocyte may not experience any torque around the y axis, the out-of-plane rotation may not affect the overall outcome significantly.

According to various embodiments, there is provided an automated apparatus for embryo biopsy. The apparatus may include an enclosure. The apparatus may further include an incubation unit which is disposed in the enclosure and which is configured to incubate an embryo. The apparatus may include an embryo manipulator setup which is disposed in the enclosure and which is configured to rotate the embryo. The apparatus may include an embryo image capturing mechanism which is disposed in the enclosure and which is configured to capture an image of the embryo in the incubation unit so as to monitor the morphology of the embryo to determine a development stage of the embryo in the incubation unit. The embryo manipulator setup may be further configured to be activated to rotate the embryo to a predetermined orientation based on a determination that the embryo is at a predetermined development stage.

According to various embodiments, the incubation unit may include a temperature control mechanism configured to control a temperature inside the incubation unit, and a carbon dioxide control mechanism configured to control an amount of carbon dioxide inside the incubation unit.

According to various embodiments, the embryo manipulator setup may include a platform and at least one three-degree-of-freedom micromanipulator attached to the platform.

According to various embodiments, the incubation unit may include an on-stage incubator.

According to various embodiments, the on-stage incubator may include a cover and a cover actuator connected to the cover. The cover actuator may be configured to actuate the opening and closing of the cover.

According to various embodiments, the cover actuator may be configured to be activated to actuate the opening of the cover based on the determination that the embryo in the on-stage incubator is at the predetermined development stage.

According to various embodiments, the on-stage incubator may be on the platform. The image capturing mechanism may be attached to the platform. The image capturing mechanism may include an imaging device and an objective lens. The objective lens may be arranged between the on-stage incubator and the imaging device.

According to various embodiments, the image capturing mechanism may be further configured to capture images of the embryo in the on-stage incubator when the embryo manipulator setup is rotating the embryo so as to monitor a position of an inner cell mass to determine an orientation of the embryo.

According to various embodiments, the apparatus may further include a multi-layer storage rack which is disposed in the enclosure and which is configured to store a plurality of on-stage incubators.

According to various embodiments, the apparatus may further include a transfer mechanism which is disposed in the enclosure and which is configured to retrieve the on-stage incubator from the multi-layer storage rack and to place the on-stage incubator on the platform.

According to various embodiments, the transfer mechanism may include a motorized two-axis translation transfer stage and a three-degree-of-freedom transfer robotic arm attached to the motorized two-axis translation transfer stage. The motorized two-axis translation transfer stage may be configured to move the three-degree-of-freedom transfer robotic arm between the multi-layer storage rack and the embryo manipulator setup. The three-degree-of-freedom robotic arm may be configured to reach into the multi-layer storage rack to pick up the on-stage incubator from the multi-layer storage rack and to position the on-stage incubator on the platform of the embryo manipulator setup.

According to various embodiments, the image capturing mechanism may include a motorized two-axis translation imaging stage disposed in the enclosure, a two-degree-of-freedom imaging robotic arm attached to the motorized two-axis translation imaging stage, and an imaging device attached to an end of the two-degree-of-freedom imaging robotic arm, The motorized two-axis translation imaging stage may be configured to move the two-degree-of-freedom imaging robotic arm between different layers of the multi-layer storage rack. The two-degree-of-freedom imaging robotic arm may be configured to move the imaging device within each layer of the multi-layer storage rack.

According to various embodiments, the transfer mechanism may be further configured to retrieve the on-stage incubator from the multi-layer storage rack and to place the on-stage incubator on the platform based on the determination that the embryo in the on-stage incubator is at the predetermined development stage.

According to various embodiments, the apparatus may further include an auxiliary image capturing mechanism. The auxiliary image capturing mechanism may be attached to the platform of the embryo manipulator setup. The auxiliary image capturing mechanism may include an auxiliary imaging device and an objective lens. The objective lens may be arranged between the platform of the embryo manipulator setup and the auxiliary imaging device.

According to various embodiments, the auxiliary image capturing mechanism may be further configured to capture images of the embryo when the embryo manipulator setup is rotating the embryo in the on-stage incubator placed on the platform so as to monitor a position of an inner cell mass to determine an orientation of the embryo.

According to various embodiments, the incubation unit may include a multi-layer incubator configured to store a plurality of embryo containers. The embryo may be hosted in an embryo container.

According to various embodiments, the multi-layer incubator may include a door and a door actuator connected to the door. The door actuator may be configured to actuate the opening and closing of the door.

According to various embodiments, the apparatus may further include a transfer mechanism which is disposed in the enclosure and which is configured to retrieve the embryo container from the multi-layer incubator so as to place the embryo container on the platform.

According to various embodiments, the transfer mechanism may include a motorized two-axis translation transfer stage and a three-degree-of-freedom transfer robotic arm attached to the motorized two-axis translation transfer stage. The motorized two-axis translation transfer stage may be configured to move the three-degree-of-freedom transfer robotic arm between the multi-layer incubator and the embryo manipulator setup. The three-degree-of-freedom robotic arm may be configured to reach into the multi-layer incubator to pick up the embryo container from the multi-layer incubator and to position the embryo container on the platform of the embryo manipulator setup.

According to various embodiments, the image capturing mechanism may include a motorized two-axis translation imaging stage disposed inside the multi-layer incubator, a two-degree-of-freedom imaging robotic arm attached to the motorized two-axis translation imaging stage, and an imaging device attached to an end of the two-degree-of-freedom imaging robotic arm. The motorized two-axis translation imaging stage may be configured to move the two-degree-of-freedom imaging robotic arm between different layers of the multi-layer incubator. The two-degree-of-freedom imaging robotic arm may be configured to move the imaging device within each layer of the multi-layer incubator.

According to various embodiments, the transfer mechanism may be further configured to retrieve the embryo container from the multi-layer incubator and place the embryo container on the platform based on the determination that the embryo in the embryo container is at the predetermined development stage.

According to various embodiments, the apparatus may further include an auxiliary image capturing mechanism. The auxiliary image capturing mechanism may be attached to the platform of the embryo manipulator setup. The auxiliary image capturing mechanism may include an auxiliary imaging device and an objective lens. The objective lens may be arranged between the platform of the embryo manipulator setup and the auxiliary imaging device.

According to various embodiments, the auxiliary image capturing mechanism may be further configured to capture images of the embryo when the embryo manipulator setup is rotating the embryo in the embryo container placed on the platform so as to monitor the position of the inner cell mass to determine an orientation of the embryo.

According to various embodiments, the platform of the embryo manipulator setup may include a heater element.

According to various embodiments, the enclosure may be an incubator box.

According to various embodiments, the apparatus may further include an embryo biopsy tool which is attached to the embryo manipulator setup, and which is configured to perform biopsy on the embryo. According to various embodiments, the embryo biopsy tool may be configured to be activated to perform biopsy based on a determination that the embryo is at a predetermined orientation. The biopsy performed may include cutting or breaching a surrounding layer of the embryo and extracting a part of the embryo from within.

According to various embodiments, the embryo manipulator setup may include two three-degree-of-freedom micromanipulators. According to various embodiments, the apparatus may further include a holding micropipette attached to one of the two three-degree-of-freedom micromanipulators, and an injection pipette attached to the other one of the two three-degree-of-freedom micromanipulators.

According to various embodiments, the apparatus may further include a processor configured to receive the captured image from the embryo image capturing mechanism, to process the captured image to determine a development stage of the embryo, and to control the embryo manipulator setup to rotate the embryo upon determination that the embryo is at the predetermined development stage. The processor may be further configured to control the biopsy tool to perform biopsy upon determination that the embryo is at the predetermined orientation.

Various embodiments have provided an all-in-one apparatus or system that reduces human intervention during the entire process from embryo incubation to embryo biopsy (e.g. from day-zero or day-three to completion), not just the biopsy process. All operations, including monitoring, recognition, and biopsy, are completed within the apparatus or system. Further, other than the embryo manipulation technique described herein, other cell manipulation and orientation device/method may also be used or performed with the apparatus or system according to the various embodiments. The embryo manipulation technique as described herein is only an example of how the embryo may be oriented to the correct position.

While the invention has been particularly shown and described with reference to specific embodiments, it should be understood by those skilled in the art that various changes, modification, variation in form and detail may be made therein without departing from the scope of the invention as defined by the appended claims. The scope of the invention is thus indicated by the appended claims and all changes which come within the meaning and range of equivalency of the claims are therefore intended to be embraced.

1. An automated apparatus for embryo biopsy, comprising:
an enclosure;
an incubation unit which is disposed in the enclosure and which is configured to incubate an embryo;
an embryo manipulator setup which is disposed in the enclosure and which is configured to rotate the embryo; and
an embryo image capturing mechanism which is disposed in the enclosure and which is configured to capture an image of the embryo in the incubation unit so as to monitor the morphology of the embryo to determine a development stage of the embryo in the incubation unit,
wherein the embryo manipulator setup is further configured to be activated to rotate the embryo to a predetermined orientation based on a determination that the embryo is at a predetermined development stage.

2. The apparatus as claimed in claim 1, wherein the incubation unit comprises a temperature control mechanism configured to control a temperature inside the incubation unit, and a carbon dioxide control mechanism configured to control an amount of carbon dioxide inside the incubation unit.

3. The apparatus as claimed in claim 1, wherein the embryo manipulator setup comprises a platform and at least one three-degree-of-freedom micromanipulator attached to the platform.

4. The apparatus as claimed in claim 3, wherein the incubation unit comprises an on-stage incubator,
wherein the on-stage incubator comprises a cover and a cover actuator connected to the cover, and
wherein the cover actuator is configured to actuate the opening and closing of the cover.

5. (canceled)

6. (canceled)

7. The apparatus as claimed in claim 4, wherein the on-stage incubator is on the platform,
wherein the image capturing mechanism is attached to the platform,
wherein the image capturing mechanism comprises an imaging device and an objective lens, and
wherein the objective lens is arranged between the on-stage incubator and the imaging device.

8. (canceled)

9. The apparatus as claimed in claim 4, further comprising a multi-layer storage rack which is disposed in the enclosure and which is configured to store a plurality of on-stage incubators, and a transfer mechanism which is disposed in the enclosure and which is configured to retrieve the on-stage incubator from the multi-layer storage rack and to place the on-stage incubator on the platform.

10. (canceled)

11. The apparatus as claimed in claim 9, wherein the transfer mechanism comprises a motorized two-axis translation transfer stage and a three-degree-of-freedom transfer robotic arm attached to the motorized two-axis translation transfer stage,
wherein the motorized two-axis translation transfer stage is configured to move the three-degree-of-freedom transfer robotic arm between the multi-layer storage rack and the embryo manipulator setup, and
wherein the three-degree-of-freedom robotic arm is configured to reach into the multi-layer storage rack to pick up the on-stage incubator from the multi-layer storage rack and to position the on-stage incubator on the platform of the embryo manipulator setup.

12. The apparatus as claimed in claim 9, wherein the image capturing mechanism comprises:
a motorized two-axis translation imaging stage disposed in the enclosure,
a two-degree-of-freedom imaging robotic arm attached to the motorized two-axis translation imaging stage, and
an imaging device attached to an end of the two-degree-of-freedom imaging robotic arm,
wherein the motorized two-axis translation imaging stage is configured to move the two-degree-of-freedom imaging robotic arm between different layers of the multi-layer storage rack, and
wherein the two-degree-of-freedom imaging robotic arm is configured to move the imaging device within each layer of the multi-layer storage rack.

13. (canceled)

14. The apparatus as claimed in claim 9, further comprising an auxiliary image capturing mechanism wherein the auxiliary image capturing mechanism is attached to the platform of the embryo manipulator setup,
wherein the auxiliary image capturing mechanism comprises an auxiliary imaging device and an objective lens, and
wherein the objective lens is arranged between the platform of the embryo manipulator setup and the auxiliary imaging device.

15. (canceled)

16. The apparatus as claimed in claim 3, wherein the incubation unit comprises a multi-layer incubator configured to store a plurality of embryo containers, and
wherein the embryo is hosted in an embryo container.

17. The apparatus as claimed in claim 16, wherein the multi-layer incubator comprises a door and a door actuator connected to the door, and
wherein the door actuator is configured to actuate the opening and closing of the door.

18. The apparatus as claimed in claim 16, further comprising a transfer mechanism which is disposed in the enclosure and which is configured to retrieve the embryo container from the multi-layer incubator so as to place the embryo container on the platform.

19. The apparatus as claimed in claim 18, wherein the transfer mechanism comprises a motorized two-axis translation transfer stage and a three-degree-of-freedom transfer robotic arm attached to the motorized two-axis translation transfer stage,
wherein the motorized two-axis translation transfer stage is configured to move the three-degree-of-freedom transfer robotic arm between the multi-layer incubator and the embryo manipulator setup, and
wherein the three-degree-of-freedom robotic arm is configured to reach into the multi-layer incubator to pick up the embryo container from the multi-layer incubator and to position the embryo container on the platform of the embryo manipulator setup.

20. The apparatus as claimed in claim 16, wherein the image capturing mechanism comprises
a motorized two-axis translation imaging stage disposed inside the multi-layer incubator,
a two-degree-of-freedom imaging robotic arm attached to the motorized two-axis translation imaging stage, and
an imaging device attached to an end of the two-degree-of-freedom imaging robotic arm,
wherein the motorized two-axis translation imaging stage is configured to move the two-degree-of-freedom imaging robotic arm between different layers of the multi-layer incubator, and
wherein the two-degree-of-freedom imaging robotic arm is configured to move the imaging device within each layer of the multi-layer incubator.

21. (canceled)

22. The apparatus as claimed in claim 16, further comprising an auxiliary image capturing mechanism wherein the auxiliary image capturing mechanism is attached to the platform of the embryo manipulator setup,
wherein the auxiliary image capturing mechanism comprises an auxiliary imaging device and an objective lens, and
wherein the objective lens is arranged between the platform of the embryo manipulator setup and the auxiliary imaging device.

23. (canceled)

24. The apparatus as claimed in claim 16, wherein the platform of the embryo manipulator setup comprises a heater element.

25. The apparatus as claimed in claim 1, wherein the enclosure is an incubator box.

26. The apparatus as claimed in claim 1, further comprising an embryo biopsy tool which is attached to the embryo manipulator setup, and which is configured to perform biopsy on the embryo.

27. (canceled)

28. The apparatus as claimed in claim 3, wherein the embryo manipulator setup comprises two three-degree-of-freedom micromanipulators, and wherein a holding micropipette is attached to one of the two three-degree-of-freedom micromanipulators and an injection pipette is attached to the other one of the two three-degree-of-freedom micromanipulators.

29. (canceled)

30. The apparatus as claimed in claim 1, further comprising a processor configured to receive the captured image from the embryo image capturing mechanism, to process the captured image to determine a development stage of the embryo, and to control the embryo manipulator setup to rotate the embryo upon determination that the embryo is at the predetermined development stage.
